(19) United States
(12) Patent Application Publication
(10) Pub. No.: US 2022/0193120 A1
(43) Pub. Date: Jun. 23, 2022

(54) MOLECULAR HYDROGEN-CONTAINING COMPOSITION FOR MAINTAINING LUNG FUNCTION IN HUMAN LUNG CANCER AND/OR IMPROVING REDUCTION IN LUNG FUNCTION ATTRIBUTED TO HUMAN LUNG CANCER

(71) Applicant: MiZ Company Limited, Kamakura-shi (JP)

(72) Inventors: Fumitake SATOH, Kamakura-shi (JP); Yusuke ICHIKAWA, Kamakura-shi (JP); Bunpei SATO, Kamakura-shi (JP); Shinichi HIRANO, Kamakura-shi (JP)

(21) Appl. No.: 17/552,193

(22) Filed: Dec. 15, 2021

(30) Foreign Application Priority Data
Dec. 17, 2020 (JP) 2020-220011

(51) Int. Cl. A61K 33/00

(52) U.S. Cl. CPC A61K 33/00

(57) ABSTRACT

The present invention provides a composition for maintaining a lung function in a human lung cancer and/or improving a reduction in lung function attributed to the human lung cancer. More specifically, the present invention provides a composition for maintaining a lung function in a human lung cancer and/or improving a reduction in lung function attributed to the human lung cancer in a subject, comprising molecular hydrogen as an active ingredient.

### RELATED APPLICATIONS

This application claims priority to Japanese Patent Application No. 2020-220011, filed on Dec. 17, 2020, the entire content of which is incorporated herein by reference.

### BACKGROUND OF THE INVENTION

### 1. Field of the Invention

The present invention provides a molecular hydrogen-containing composition for maintaining the lung function in a human lung cancer and/or improving a reduction in lung function attributed to the human lung cancer.

### 2. Description of the Related Art

A WHO estimation shows that the number of deaths caused by lung cancer occupies 17% of the total number of deaths caused by all types of cancer, and is the largest thereof, and 1,300,000 people yearly die of lung cancer in the world. Lung cancer reduces the lung function, and also impairs the QOL of cancer patients due to bloody sputum, chronic severe cough, wheezing, chest pain, shortness of breath, and respiratory distress. However, relaxation of these symptoms without side effects is difficult in the current medicine and pharmaceutical treatments, which provide poor outcomes. Although an oxygen therapy to inhale high concentration oxygen is used against respiratory distress, the high concentration oxygen generates a larger amount of reactive oxygen which attack substances forming cells, and thus promotes a reduction in lung function.

Hydrogen, the active ingredient of the present invention, can eliminate hydroxyl radicals having the highest oxidation activity among such reactive oxygens, and convert those into water molecules (Fukai Yuh “Suiso Bunshi wa Kanari Sugoi”, Kobunsha Co., Ltd., 2017, p. 24). Because hydrogen acts as an antioxidant which eliminates hydroxyl radicals, there are many reports that administration of hydrogen can cure diseases associated with oxidative stress.

Because hydrogen is a gas and is directly transported to the lung after inhaled, effects against respiratory diseases can be expected. The following reports are known so far: a report that metastasis in lung cancer model mice to which Lewis Lung Carcinoma was transplanted was suppressed by inhalation of low concentration (3.5%) hydrogen (Japanese Patent No. 6781485, Example 1), and a report that bronchial stenoses of obstructive bronchiolitis in respiratory disease model mice were improved by drinking of hydrogen water (Japanese Patent No. 6628449). However, small animals such as disease model mice and human are different species and thus are different in not only the appearance but also DNA. Thus, the improving effects of hydrogen demonstrated in animal tests are not always guaranteed in human (Fukai Yuh, op. cit., p. 57).

The medical treatment effects of hydrogen significantly depend on the administration method, namely, inhalation of hydrogen gas or drinking of hydrogen water. Accordingly, selection of the administration method depending on the symptoms of diseases should be examined in detail as one of problems of medical treatments with hydrogen in the future ((Fukai Yuh, op. cit., p. 92).

Furthermore, because high concentration hydrogen molecules are explosive, hydrogen having a low concentration equal to or less than the explosion limit should be used in treatments and maintenance. However, it should also be verified whether hydrogen having a low concentration equal to or less than the explosion limit demonstrates sufficient maintenance and improving effects against each of the diseases.

Although there are reports about the improving effects against human lung cancer, such improving effects in these reports are all derived from use of a high concentration (66% or 67%) hydrogen gas generator, and risks of explosion remain still ([Junji Akagi et al., “Hydrogen gas activates coenzyme Q10 to restore exausted CD8+ T cells, especially PD-1+Tim+terminal CD8+ T cells, leading to better nivolumab outcomes in patients with lung cancer.” ONCOLOGY LETTERS 20:258, 2020], [Ji-Bing Chen, et al., “Real world survey” of hydrogen-controlled cancer: a follow-up reports of 82 advanced cancer patients. Medical Gas Research, 2019; 9(3):115-121.], and [Ji-Bing Chen, et al., “Hydrogen therapy can be used to control tumor progression and alleviates the adverse events of medication in patients with advanced non-small cell kung cancer.” Medical Gas Research, 2020; 10(2):75-80]).

### SUMMARY OF THE INVENTION

To solve the above problems, the present invention reports, for the first time ever, that the lung function in a human lung cancer was maintained and/or a reduction in lung function attributed to the human lung cancer was improved with a low hydrogen concentration equal to or less than the explosion limit (e.g., equal to or less than the detonation limit of 18.5%).

That is, the present invention encompasses the following characteristics:

(1) A composition for maintaining a lung function in a human lung cancer and/or improving a reduction in lung function attributed to the human lung cancer, comprising molecular hydrogen as an active ingredient, wherein a gas comprising the molecular hydrogen has a hydrogen concentration of higher than zero (0) and not higher than 18.5% by volume.

(2) The composition according to (1), wherein the lung function is breathing, ventilatory function, and/or a function associated therewith.

(3) The composition according to (1), wherein a cause of the reduction in lung function attributed to the human lung cancer is a lung cancer, pneumonia attributed to a lung cancer, emphysema attributed to a lung cancer, and/or a disease associated therewith.

(4) The composition according to any one of (1) to (3), wherein the lung cancer is a lung cancer caused by smoking.

(5) The composition according to any one of (1) to (4), wherein the composition comprising the molecular hydrogen as the active ingredient is fed to a subject by inhalation of the composition using a hydrogen gas supply apparatus.

(6) A method for administering a composition comprising molecular hydrogen as an active ingredient for the purpose of maintaining a lung function in a human lung cancer and/or improving a reduction in lung function attributed to the human lung cancer, wherein a gas comprising the molecular hydrogen has a hydrogen concentration of higher than zero (0) and not higher than 18.5% by volume.

(7) The method according to (6), wherein the lung function is breathing, ventilatory function, and/or a function associated therewith.

(8) The method according to (6), wherein a cause of the reduction in lung function attributed to the human lung cancer is a lung cancer, pneumonia attributed to a lung cancer, emphysema attributed to a lung cancer, and/or a disease associated therewith.

(9) The method according to any one of (1) to (8), wherein the lung cancer is a lung cancer caused by smoking.

(10) The method according to any one of (1) to (9), wherein the composition comprising the molecular hydrogen as the active ingredient is fed to a subject by inhalation of the composition using a hydrogen gas supply apparatus.

The lung function in the human lung cancer can be maintained and/or a reduction in lung function attributed to the human lung cancer can be improved even with a low hydrogen concentration equal to or less than the explosion limit.

### Advantageous Effects of Invention

The present invention provides a composition and/or a method for maintaining a lung function in a human lung cancer and/or improving a reduction in lung function attributed to the human lung cancer, the composition and/or the method comprising molecular hydrogen as an active ingredient.

### DETAILED DESCRIPTION OF THE PREFERRED EMBODIMENTS

The present invention will be described in more detail below.

1. Composition or Method for Maintaining Lung Function in Human Lung Cancer and/or Improving Reduction in Lung Function Attributed to Human Lung Cancer

The present invention provides a composition for maintaining a lung function in a human lung cancer and/or improving a reduction in lung function attributed to the human lung cancer, comprising molecular hydrogen as an active ingredient.

In the present specification, the term “lung function in the human lung cancer” is a lung cancer, pneumonia attributed to a lung cancer, emphysema attributed to a lung cancer, and/or a disease associated therewith.

In the present specification, the term “function associated therewith” in “the lung function is breathing, ventilatory function, and/or a function associated therewith” includes foreign substances elimination action of ciliated epithelia, foreign substances elimination action by mucus, ventilatory control by autonomic nerves, i.e., sympathetic nerves and/or parasympathetic nerves, chemical breath control which controls ventilation by sensing the levels of oxygen, carbon dioxide, and the pH, breath control accompanied by the body temperature, breath control by acceleration of metabolism accompanied by exercise or the like, voluntary breath control, reflex breath control, and alveolar diffusion disorder, for example.

In the present specification, the term “cause of the reduction in lung function attributed to the human lung cancer” includes lung cancers, pneumonia attributed to lung cancers, emphysema attributed to lung cancers, and/or diseases associated therewith.

In the present specification, the term “disease associated therewith” in “lung cancer, pneumonia attributed to a lung cancer, emphysema attributed to a lung cancer, and/or a disease associated therewith” includes restrictive lung diseases such as chronic obstructive pulmonary disease (COPD), asthma, interstitial pneumonia, pulmonary fibrosis, and adhesion, for example.

In the present specification, the term “subject” includes mammalians such as primates including humans, pet animals such as dogs and cats, and ornamental animals such as zoo animals. Preferred subjects are humans.

The term “human” includes a patient having a disease included in the present invention. However, the term “human” does not include disease model animals used in animal tests, such as mice, rats, dogs, cats, monkeys, pigs, fish, and insects. The disease model animals have diseases basically different from those of patients (including mammalians (pet animals) diagnosed by veterinarians) diagnosed by medical practitioners, such as medical doctors and veterinarians, in clinical sites such as medical institutions.

In the present specification, “hydrogen,” the active ingredient of the composition of the present invention, is molecular hydrogen (i.e., gaseous hydrogen or hydrogen gas) and is simply referred to as “hydrogen” or “hydrogen gas” unless otherwise specified. Additionally, the term “hydrogen” used in the present specification refers to a molecular formula of H₂, D₂ (deuterium), or HD (deuterated hydrogen) or a gas mixture thereof. D₂ is expensive but known to have a stronger superoxide eliminating effect than that of H₂. Hydrogen that can be used in the present invention is H₂, D₂ (deuterium), HD (deuterated hydrogen), or a gas mixture thereof, preferably H₂. Alternatively, D₂, and/or HD can be used instead of H₂ or in a mixture with H₂.

Preferred embodiments of the composition of the present invention are gases or liquids containing molecular hydrogen, preferably gases containing molecular hydrogen.

The gases containing molecular hydrogen are preferably air containing hydrogen gas or a mixed gas containing hydrogen gas and oxygen gas. The concentration of hydrogen gas in a gas containing molecular hydrogen is higher than zero (0) and not higher than 18.5% by volume, for example, 0.5% to 18.5% by volume, preferably 1% to 10% by volume, for example, 2% to 8% by volume, 2% to 9% by volume, 2% to 10% by volume, 3% to 6% by volume, 3% to 7% by volume, 3% to 8% by volume, 3% to 9% by volume, 3% to 10% by volume, 4% to 5% by volume, 4% to 6% by volume, 4% to 7% by volume, 4% to 8% by volume, 4% to 9% by volume, 4% to 10% by volume, 5% to 8% by volume, 5% to 9% by volume, 5% to 10% by volume, 6% to 7% by volume, 6% to 8% by volume, 6% to 9% by volume, 6% to 10% by volume, and the like. In the present invention, higher hydrogen gas concentration (but equal to or less than the explosion limit) or larger daily hydrogen doses tend to be associated with greater effects of maintaining or improving the disease of the human or the animal. In the present invention, a hydrogen gas supply apparatus which generates and emits hydrogen in the safety concentration above should be used.

Because hydrogen is a gas without any accompanied pharmacological side effects, the inhalation time is not limited. The action mechanism of hydrogen is to eliminate hydroxyl radicals generated inside cells, and particularly inside mitochondria to protect cells and substances forming cells from oxidative stress or oxidation. Accordingly, the estimated inhalation amount of hydrogen needed to eliminate hydroxyl radicals and peroxynitrite to protect cells from oxidative stress can be calculated from the number of cells forming a body or the number of mitochondria inside cells. To be noted, a larger amount of hydrogen should be inhaled to sufficiently feed hydrogen to the inside of a relatively large organism such as a liver.

From such a viewpoint, the inhalation time of hydrogen is preferably within 1 hour daily. More preferably, the daily inhalation time is preferably any time selected from the range of 6 hours or less, such as 2 hours or less, 3 hours or less, 4 hours or less, and 5 hours or less. Still more preferably, the daily inhalation time is preferably any time selected from the range of 12 hours or less, such as 7 hours or less, 8 hours or less, 9 hours or less, 10 hours or less, and 11 hours or less. Further still more preferably, the daily inhalation time is preferably any time selected from the range of 24 hours or less, such as 13 hours or less, 14 hours or less, 15 hours or less, 16 hours or less, 17 hours or less, 18 hours or less, 19 hours or less, 20 hours or less, 21 hours or less, 22 hours or less, and 23 hours or less.

Hydrogen is a flammable and explosive gas, and use of a hydrogen gas having a hydrogen concentration of 18.5% or more should be avoided because it involves not only explosion but also detonation. Accordingly, it is preferable to add hydrogen to the composition of the present invention under conditions safe for subjects such as humans and administer the mixture to subjects to maintain the lung function in the human lung cancer and/or to improve a reduction in lung function attributed to the human lung cancer.

When a gas other than hydrogen gas is air, the air concentration is in the range of, for example, 81.5% to 99.5% by volume. When a gas other than hydrogen gas is a gas containing oxygen gas, the oxygen gas concentration is in the rage of, for example, 21% to 99.5% by volume. As another main gas, for example, nitrogen gas can be further added.

The liquids containing molecular hydrogen are specifically aqueous liquids containing a dissolved hydrogen gas. Examples of the aqueous liquids used herein include, but are not limited to, water (e.g., purified water, sterilized water), physiological saline, buffer solutions (e.g., buffer solutions of pH 4 to 7.4), drip infusion solutions, fluid infusion solutions, injection solutions, and drinks (e.g., tea drinks such as green tea and black tea, fruit juice, green juice, vegetable juice). Examples of the hydrogen concentration in a liquid containing molecular hydrogen include, but are not limited to, 1 to 10 ppm, preferably 1.2 to 9 ppm, for example, 1.5 to 9 ppm, 1.5 to 8 ppm, 1.5 to 7 ppm, 1.5 to 6 ppm, 1.5 to 5 ppm, 1.5 to 4 ppm, 2 to 10 ppm, 2 to 9 ppm, 2 to 8 ppm, 2 to 7 ppm, 2 to 6 ppm, 2 to 5 ppm, 3 to 10 ppm, 3 to 9 ppm, 3 to 8 ppm, 3 to 7 ppm, 4 to 10 ppm, 4 to 9 ppm, 4 to 8 ppm, 4 to 7 ppm, 5 to 10 ppm, 5 to 9 ppm, 5 to 8 ppm, and 5 to 7 ppm.

A gas or a liquid containing molecular hydrogen is formulated to provide a predetermined hydrogen gas concentration and then with the same, for example, a pressure-resistant container (e.g., a stainless cylinder, an aluminum can, a pressure-resistant plastic bottle [e.g., a pressure-resistant PET bottle] and a plastic bag preferably having the inside laminated with an aluminum film, or an aluminum bag) is filled. Aluminum has the property of unlikely allowing hydrogen molecules to pass therethrough. Alternatively, a gas containing molecular hydrogen or a liquid containing molecular hydrogen may be produced in situ before use by using an apparatus such as a hydrogen gas generating apparatus, a hydrogen water generating apparatus, or a hydrogen gas adding apparatus such as a known or commercially available hydrogen gas supply apparatus (an apparatus for generating a gas containing molecular hydrogen), a hydrogen adding device (an apparatus for hydrogen water generation), or a non-destructive hydrogen adding apparatus (e.g., an apparatus for non-destructively adding hydrogen gas into a bag for a biocompatible solution such as a drip infusion solution).

The hydrogen gas supply apparatus enables hydrogen gas generated from a reaction of a hydrogen generating agent (e.g., metallic aluminum, magnesium hydride) and water to be mixed with a diluent gas (e.g., air, oxygen) in a predetermined ratio (refer to Japanese Patent No. 5228142, etc.). Or, the hydrogen gas supply apparatus mixes hydrogen gas generated utilizing electrolysis of water with a diluent gas such as oxygen or air (refer to Japanese Patent No. 5502973, Japanese Patent No. 5900688, etc.). Thus, a gas containing molecular hydrogen at a hydrogen concentration in the range of, for example, 0.5% to 18.5% by volume can be prepared.

The hydrogen adding device is an apparatus that generates hydrogen by using a hydrogen generating agent and a pH modifier and dissolving the hydrogen in a biocompatible solution such as water (refer to Japanese Patent No. 4756102, Japanese Patent No. 4652479, Japanese Patent No. 4950352, Japanese Patent No. 6159462, Japanese Patent No. 6170605, Japanese Patent Laid-open No. 2017-104842, etc.). Examples of a mixture of a hydrogen generating agent and a pH modifier include metallic magnesium and a strongly acidic ion exchange resin or an organic acid (e.g., malic acid, citric acid) and a metallic aluminum powder and a calcium hydroxide powder. With these mixtures, a liquid containing molecular hydrogen at a dissolved hydrogen concentration of, for example, approximately 1 to 10 ppm can be prepared.

The non-destructive hydrogen adding apparatus is an apparatus or a device that adds hydrogen gas to a commercially available biocompatible solution such as a drip infusion solution (e.g., enclosed in a hydrogen-permeable plastic bag such as a polyethylene bag) from the outside of a package and is commercially available from, for example, MiZ Company Limited (http://www.e-miz.co.jp/technology.html). This apparatus can dissolve hydrogen in a biocompatible solution aseptically until the equilibrium concentration is reached, by immersing a bag containing the biocompatible solution in saturated hydrogen water, so that hydrogen is permeated into the bag. The apparatus is composed of, for example, an electrolytic bath and a water bath, and water in the water bath is circulated in the electrolytic bath and the water bath to generate hydrogen by electrolysis. Or, a simplified, disposable device can be used for a similar purpose (refer to Japanese Patent Laid-open No. 2016-112562, etc.). This device has a biocompatible solution-containing plastic bag (a hydrogen-permeable bag, for example, a polyethylene bag) and a hydrogen generating agent (e.g., metallic calcium, metallic magnesium/cation exchange resin) incorporated in an aluminum bag, and the hydrogen generating agent is wrapped with, for example, a non-woven fabric (e.g., steam-permeable non-woven fabric). Hydrogen generated by wetting the hydrogen generating agent wrapped with a non-woven fabric with a small amount of water, such as a steam, is dissolved in a biocompatible solution non-destructively and aseptically.

Or, a purified hydrogen gas cylinder, a purified oxygen gas cylinder, or a purified air cylinder may be provided to produce a gas or a liquid containing molecular hydrogen which is adjusted to provide a predetermined hydrogen concentration or a predetermined oxygen or air concentration.

The gas containing molecular hydrogen or the liquid (such as water (such as purified water and sterilized water), physiological saline, or drop infusion solutions) containing molecular hydrogen prepared using the above-mentioned apparatuses or the devices can be administered orally or parenterally to subjects having a lung function in the lung cancer subjects before, during, or after surgery.

Other embodiments of the composition of the present invention include dosage forms (e.g., tablets, capsules) prepared to be orally administered to (or ingested by) subjects, which contain a hydrogen generating agent that enables hydrogen to be generated in the gastrointestinal tract. The hydrogen generating agent preferably comprises, for example, components approved as food or food additives.

When the composition of the present invention comprises molecular hydrogen as an active ingredient, examples of the method of administering the composition to subjects include administration by inhalation, suction or the like. For example, transpulmonary administration is preferred. When a liquid containing molecular hydrogen is contained as an active ingredient, oral or intravenous administration (including drip infusion) is preferred. When a gas is inhaled, the gas can be inhaled from the mouth or the nose to the lung via a nasale cannula, a mask-like device covering the mouth and the nose, or a hydrogen feedable chamber such as a chamber, and then can be delivered to the whole body by blood.

The liquid containing molecular hydrogen to be orally administered may be administered to subjects as a cooled liquid or a liquid stored at room temperature. Hydrogen is dissolved in water at a concentration of approximately 1.6 ppm (1.6 mg/L) at room temperature and under a normal pressure, and the difference in solubility due to temperature is known to be relatively small. Or, when a liquid containing molecular hydrogen is, for example, in the form of a drip infusion solution or an injection solution containing hydrogen gas prepared using the above-described non-destructive hydrogen adding apparatus, the liquid may be administered to subjects by parenteral routes, such as intravenous or intraarterial administration.

One dose or multiple doses (e.g., two to three doses) per day of a gas containing molecular hydrogen at the above-mentioned hydrogen concentrations or a liquid containing molecular hydrogen at the above-mentioned dissolved hydrogen concentrations can be administered to humans for a period of one week to three months or longer, for example, one week to six months or longer (e.g., one year or longer, two years or longer). When a gas containing molecular hydrogen is administered, the gas is preferably inhaled for at least 30 minutes per dose. Because the improving effect becomes higher with a longer inhalation time, the gas can be administered for, for example, 30 minutes to one hour, one hour to two hours, two hours to three hours, or longer. Additionally, when a gas containing molecular hydrogen is administered in a transpulmonary manner by inhalation or suction, the gas can be administered to subjects under an atmospheric pressure environment, or, for example, under a high atmospheric pressure in the range exceeding a standard atmospheric pressure (i.e., approximately 1.013 atm) and not higher than 7.0 atm, for example, under a high atmospheric pressure environment in the range of 1.02 to 7.0 atm, preferably in the range of 1.02 to 5.0 atm, more preferably in the range of 1.02 to 4.0 atm, yet more preferably in the range of 1.02 to 1.35 atm (including the gas containing molecular hydrogen).

2. Method for Maintaining Lung Function in Human Lung Cancer and/or Improving Reduction in Lung Function Attributed to Human Lung Cancer

Furthermore, the present invention provides a method for accelerating recovery or improvement of a subject having a lung cancer from invasion in surgery and/or a symptom associated with the surgery using the composition comprising the molecular hydrogen as the active ingredient.

The composition comprising the molecular hydrogen, maintenance of the lung function in the human lung cancer, and/or the symptoms associated with the lung function attributed to the human lung cancer, the dose, the administration method and the like are as described in the above 1.

In the method of the present invention, a gas containing molecular hydrogen (preferably, air or oxygen) at higher than zero (0) and not higher than 18.5% by volume, for example, 0.5% to 18.5% by volume, 2% to 10% by volume, 2% to 9% by volume, 2% to 8% by volume, 3% to 10% by volume, 3% to 9% by volume, 3% to 8% by volume, 3% to 7% by volume, 3% to 6% by volume, 4% to 10% by volume, 4% to 9% by volume, 4% to 8% by volume, 4% to 7% by volume, 4% to 6% by volume, 4% to 5% by volume, 5% to 10% by volume, 5% to 9% by volume, 5% to 8% by volume, 6% to 10% by volume, 6% to 9% by volume, 6% to 8% by volume, 6% to 7% by volume, or the like, preferably 5% to 10% by volume, 5% to 8% by volume, for example, 6% to 10% by volume, 6% to 8% by volume, 6% to 7% by volume, or the like can be inhaled or sucked by subjects for, for example, one to three hours or longer per day and can be continued for, for example, one to three months or longer, four to seven months or longer, one to three years or longer.

Alternatively, in the method of the present invention, for example, the standard concentration for the molecular hydrogen-containing liquid to be administrated to the subject is more than 0 ppm and 1.6 ppm or less. Preferably, the concentration is 2.0 to 5.0 ppm, 2.0 to 6.0 ppm, 2.0 to 7.0 ppm, or 2.0 to 8.0 ppm, or 2 to 9 ppm. More preferably, the concentration is 3.0 to 7.0 ppm, 3.0 to 8.0 ppm, 3.0 to 9.0 ppm, 3.0 to 10 ppm, 4.0 to 7.0 ppm, 4.0 to 8.0 ppm, 4.0 to 9.0 ppm, 4.0 to 10 ppm, 5.0 to 7.0 ppm, 5.0 to 8.0 ppm, 5.0 to 9.0 ppm, 5.0 to 10 ppm, 3.0 to 7.0 ppm, 4.0 to 8.0 ppm, 5 to 7.0 ppm, 5.0 to 8.0 ppm, or 5.0 to 9.0 ppm. Still more preferably, the concentration is more than 0.0 ppm and 10 ppm or less, 1.0 to 10 ppm, 1.5 to 10 ppm, 2.0 to 10 ppm, 3.0 to 10 ppm, 4.0 to 10 ppm, 5.0 to 10 ppm, 6.0 to 10 ppm, or 7.0 to 10 ppm.

200 to 500 mL per dose for intravenous administration or, for example, 500 to 1000 mL per dose for oral administration of a liquid containing molecular hydrogen can continue to be administered to subjects for, for example, 0.5 to three months or longer, four to seven months or longer, one to three years or longer.

The method of the present invention may further be used in combination with a therapeutic agent used for the treatment of the lung function of the subject having the lung cancer, if necessary. Such a combination use is expected to increase levels of maintenance and/or improvement of the lung function of the subject having the lung cancer.

### EXAMPLE

The present invention is explained more specifically with reference to the following example. However, the example is not intended to limit the scope of the present invention. The diagnosis by doctors also involves personal intuitive opinions based on their experiences, rather than is based on only scientific grounds. Thus, the present invention is not always limited by the diagnosis by doctors.

### Example 1

### <Case 1 of Lung Function of Subject Having Lung Cancer by Hydrogen Inhalation>

A 55-year-old male of stage IV lung cancer, who had smoking habit, was discharged from hospital on his own will in July, 2020. When discharged, he had complications of interstitial pneumonia, emphysema, and multiple metastasis attributed to the lung cancer. An inflammation reaction such as colitis was observed although its association with the lung cancer was unclear. The lung cancer was originated from the right lung. The man was subjected to administration of an anticancer agent and a radiation therapy, but an improvement of symptoms was not recognized. In a radiograph, white shadows unique to the lung cancer were spread from the right lung. A breadth of shadows unique to pneumonia was observed in an image from chest computed tomographic (CT) examination. Although the male took a pneumonia drug, any trend of improvement was not observed. The doctors determined that it was difficult to attain any improvement by the present medical therapeutic methods.

In the first half of October in 2020, the male was nearly confined to bed, and ate some times and did not eat some times due to his inconsistent appetite. He was treated with oxygen inhalation because the ventilatory function of the lung was reduced.

On Oct. 21, 2020, inhalation of the hydrogen gas was started using a hydrogen gas generator (Jobs-α, hydrogen concentration: about 4% to 5%, the amount of 100% hydrogen to be generated: 200 ml/min) manufactured by MiZ Company Limited (Ofuna, Kamakura). The male was treated with inhalation of hydrogen gas for at least 3 hours per day.

In Week 1 at the start of inhalation, inhalation performed for 5 hours per day because the male largely spent the daytime while laying. The hydrogen gas could be inhaled comfortably with no discomfort.

In Week 2, breathing became easier, and the state of breathing became better. The amount of oxygen needed for oxygen inhalation was reduced. The lung was X-rayed, and any significant change was not observed in the image.

In Week 3, the man was subjected to chest CT examination. Surprisingly to the doctors, the shadows derived from pneumonia before hydrogen inhalation completely disappeared in the CT image. The amount of oxygen needed for oxygen inhalation was reduced compared to that in the previous week, showing that the amount of oxygen needed was likely to be gradually decreased. From the CT image diagnosis and the reduced amount of oxygen needed, the doctors determined that the lung ventilatory function was improved compared to that one month ago. The complicating inflammation reaction such as colitis was also improved.

### Example 2

### <Case 2 of Lung Function of Subject Having Lung Cancer by Hydrogen Inhalation>

A 70-year-old male had surgery of colorectal cancer in 2018. Thereafter, the male was periodically examined for follow-up. In March of 2020, an increase in numeric value of tumor markers was verified. CT and PET revealed that the lung had a cancer. Reductions in lung capacity and breathing function were also verified. From the day of examination, the male was treated with hydrogen gas inhalation for 3 to 6 hours per day using a hydrogen gas generator (Jobs-mini, hydrogen concentration: about 1% to 2%, the amount of 100% hydrogen to be generated: 30 ml/min). Because surgery of the lung cancer was scheduled, a pre-surgery examination was performed one month later from the start of inhalation, and showed that the lung cancer disappeared. The tumor markers showed normal numeric values again, and the lung capacity and the breathing function also showed normal values again.

The present invention can maintain the lung function in the human lung cancer and/or improve the reduction in lung function attributed to the human lung cancer by administering the composition comprising molecular hydrogen.

What is claimed is:

1. A method for maintaining lung function of a human suffering from lung cancer and/or improving a reduction in lung function caused by lung cancer in a human, comprising administering to the human a gas composition comprising an effective amount of molecular hydrogen
wherein the gas composition comprising the molecular hydrogen has a hydrogen concentration of higher than zero (0) and not higher than 18.5% by volume.

2. The method according to claim 1, wherein the lung function is breathing, ventilatory function, and/or a function associated therewith.

3. The method according to claim 1, wherein the reduction in lung function is pneumonia caused by a lung cancer, emphysema caused by a lung cancer, and/or a disease associated with a lung cancer.

4. The method according to claim 1, wherein the lung cancer is a lung cancer caused by smoking.

5. The method according to claim 1, wherein the gas composition is supplied for inhalation of the composition to a subject by using a hydrogen gas supply apparatus.
